(19) United States
(12) Patent Application Publication
(10) Pub. No.: US 2006/0235255 A1
(43) Pub. Date: Oct. 19, 2006

(54) Double bond hydroisomerization process

(76) Inventors: Robert J. Gartside, Summit, NJ (US); Thomas P. Skourlis, Basking Ridge, NJ (US); Robert E. Trubac, Ridgewood, NJ (US); Hassan Kaleem, Franklin Park, NJ (US)

Correspondence Address: KENNETH H. JOHNSON, P.O. BOX 630708, HOUSTON, TX, 77263, US

(21) Appl. No.: 11/107,667

(22) Filed: Apr. 15, 2005

(51) Int. Cl. C07C 5/25; C07C 5/23

(52) U.S. Cl. 585/664

(57) ABSTRACT

A process and apparatus are disclosed for hydroisomerizing a mixed C4 olefin stream in a catalytic distillation column in order to increase the concentration of 2-butene and minimize the concentration of 1-butene, while concurrently minimizing the production of butanes. In one embodiment, carbon monoxide is introduced into the double bond hydroisomerization reactor along with hydrogen. In another embodiment, hydrogen, and optionally also carbon monoxide, is introduced at multiple locations along the double bond hydroisomerization reactor. The invention is particularly useful in preparing C4 feed streams for metathesis reactions.

### FIELD OF THE INVENTION

The present invention generally relates to double bond hydroisomerization reactions, and more particularly to a process and apparatus for improving the selectivity of double bond hydroisomerization of 1-butene to 2-butene.

### BACKGROUND OF THE INVENTION

In many processes it is desirable to have isomerization of double bonds within a given molecule. Double bond isomerization is the movement of the position of the double bond within a molecule without changing the structure of the molecule. This is different from skeletal isomerization where the structure changes (most typically representing the interchange between the iso form and the normal form). Skeletal isomerization proceeds by a completely different mechanism than double bond isomerization. Skeletal isomerization typically occurs using a promoted acidic catalyst.

There are two basic types of double bond isomerization, namely hydroisomerization and non-hydroisomerization. The former uses small quantities of hydrogen over noble metal catalysts (such as Pt or Pd) and occurs at moderate temperatures while the latter is hydrogen free and typically employs basic metal oxide catalysts at higher temperatures.

Double bond hydroisomerization of 1-butene to 2-butene can be a side reaction that occurs in a fixed bed as part of a selective hydrogenation step in which butadiene is converted to butene, or “on purpose” in a separate fixed bed reactor following a selective hydrogenation step. Double bond hydroisomerization at moderate temperatures is mostly used to maximize the interior olefin (2-butene for example as opposed to 1-butene) since the thermodynamic equilibrium favors the interior olefin at lower temperatures. This technology is used when there is a reaction that favors the interior olefin over the alpha olefin. Ethylenolysis of 2-butene to make propylene is such a reaction. The ethylenolysis (metathesis) reaction is 2-butene+ethylene→2 propylenes.

Double bond hydroisomerization does not however occur to any great extent in streams that contain highly unsaturated components (acetylenes or dienes). Typical feedstocks are steam cracker C4's or fluid catalytic cracker C4 steams. For steam cracker C4 streams, butadiene as well as ethyl and vinyl acetylene are usually present. Butadiene is present in large quantities, e.g. around 40% of the C4 fraction. A selective hydrogenation unit is utilized to turn the butadiene into butene if butadiene is not desired as a product and also to hydrogenate the ethyl and vinyl acetylenes. If butadiene is desired as a product, it can be removed by extraction or another suitable process. The exit butadiene from extraction is typically on the order of 1 wt % of the C4 stream or less.

To reduce butadiene to low levels (<1000 ppm), hydrogenation is required. Two fixed bed reactors are typically employed in a hydrogenation process if butadiene is present in substantial quantities, or a single fixed bed reactor is employed if the concentration is lower (ca. butadiene removal by extraction). In either case, depending upon how the second or “trim” reactor is operated, varying degrees of isomerization of 1-butenes to 2-butenes occurs in this second reactor. In addition, some hydrogenation of the butenes to butanes occurs, representing losses of olefins.

The double bond hydroisomerization reaction of butene is represented by:
1-C4H8→2-C4H8
There is no hydrogen uptake in this reaction. However, a slight amount of hydrogen is required for the process to facilitate the reaction taking place on the catalyst. It is assumed that hydrogen is present on the surface of the catalyst and maintains it in an “active” form.

The hydrogenation of butadiene occurs as follows: 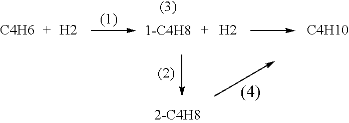
The principal product of butadiene hydrogenation is 1-butene. However as the concentration of butadiene is reduced, isomerization reactions begin to take place, forming 2-butene. This accelerates as butadiene approaches low values (<0.5%) and the hydrogenation of butenes to butanes becomes significant. It is well established that these reactions occur in varying proportion over typical hydrogenation catalysts (Group VIII) metals such as Pd, Pt, Ni. It is further well known that the relative rates of forward reactions (1, 2, 3, 4) are in the relative ratio of 100:10:1:1. This shows that the principal product of butadiene hydrogenation is 1-butene. As butadiene is hydrogenated and a substantial quantity of 1-butene is formed, it continues to react in the presence of hydrogen to form 2-butene (double bond hydroisomerization) and butane (continued hydrogenation). The double bond hydroisomerization reaction is preferred. The rate of hydrogenation of 1-butene to butane or 2-butene to butane occurs but at a lower rate. Reaction selectivity is in proportion to the rates of reaction. In the double bond hydroisomerization of 1-butene to 2-butene, typically 90% of the 1-butene converted is to 2-butene and 10% is to butane. Under these conditions, minimal skeletal isomerization occurs (1- or 2-butene to isobutylene).

In a double bond hydroisomerization process, the hydrogen rate to the reactor must be sufficient to maintain the catalyst in the active double bond hydroisomerization form because hydrogen is lost from the catalyst by hydrogenation, especially when butadiene is contained in the feed. The hydrogen rate must be adjusted such that there is sufficient amount to support the butadiene hydrogenation reaction and replace hydrogen lost from the catalyst, but the amount of hydrogen should be kept below that required for hydrogenation of butenes.

Hydroisomerization and hydrogenation reactions in fixed bed reactors are described in U.S. Pat. No. 3,531,545. This patent discloses a process and method for double bond isomerization consisting of mixing a hydrocarbon stream containing 1-olefins and at least one sulfur-containing compound with hydrogen, heating the mixed hydrocarbon/hydrogen stream to reaction temperatures, contacting the stream with a noble metal catalyst, and then recovering the 2-olefins as a product. The process described in this patent utilizes sulfur as an additive to reduce the hydrogenation tendency of the catalyst and thus increase hydroisomerization. Sulfur is shown to be either present in the feed, added to the feed, or added to the hydrogen stream.

It is known to use double bond hydroisomerization to convert 2-butene to 1-butene. In U.S. Pat. No. 5,087,780, “Hydroisomerization Process”, assigned to Chemical Research & Licensing Company, a process is disclosed for the isomerization of butenes in a mixed hydrocarbon stream containing 1-butene, 2-butene and small amounts of butadiene in which the mixed hydrocarbon stream is fed to a distillation column reactor containing an alumina supported palladium oxide catalyst as a distillation structure. As 1-butene is produced it is distilled off, upsetting the equilibrium and allowing for a greater than equilibrium amount of 1-butene to be produced. Additionally, any butadiene in the feed is hydrogenated to butenes. The bottoms, which is rich in 2-butene, may be recycled to the reactor column for more complete conversion of 2-butene to 1-butene. Alternatively, a portion or essentially all of the bottoms, substantially free of butadiene, may be used for feed to an HF alkylation unit.

Double bond isomerization reactions of C4 hydrocarbons can also occur over basic metal oxide catalysts. In this case, the process is not hydroisomerization but simple double bond isomerization. This reaction occurs in the vapor phase at high temperatures (>200 deg. C.) without the addition of hydrogen and should not be confused with double bond hydroisomerization that occurs primarily in the liquid phase at lower temperatures (<150 deg. C.).

As an alternative to a process using a fixed bed reactor, double bond hydroisomerization can be practiced in a catalytic distillation reactor. In U.S. Pat. No. 6,242,661, “Process for the Separation of Isobutene from Normal Butenes”, assigned to Catalytic Distillation Technologies, isobutene and isobutane are removed from a mixed C4 hydrocarbon stream which also contains 1-butene, 2-butene and small amounts of butadiene. A catalytic distillation process is used in which a particulate supported palladium oxide catalyst isomerizes 1-butene to 2-butene. Isomerization is desired because 2-butene can be separated from isobutene more easily than 1-butene. As 2-butene is produced, it is removed from the bottom of the column, upsetting the equilibrium and allowing for a greater than equilibrium amount of 2-butene to be produced. Butadiene in the feed stream is hydrogenated to butene.

Double bond hydroisomerization processes can be combined with metathesis. The metathesis reaction in this case typically is the reaction between ethylene and 2-butene to form propylene. The presence of 1-butene in the feed results in reduced selectivity and thus lower propylene production. In addition, in metathesis of 2-butenes with ethylene to form propylene, it is desired to remove isobutylene and isobutane to minimize the flow of these components through the metathesis reaction system since they are essentially inerts.

The amount of 2-butene can be maximized from a C4 stream (after butadiene removal) by double bond hydroisomerization. In the design of a metathesis unit, this can be accomplished by passing the feed through a fixed bed hydrosiomerization reactor with sufficient hydrogen as described above. Isobutylene and isobutane removal can then be accomplished by fractionation. As an alternative, a catalytic distillation-deisobutenizer (CD-DeIB) can be employed. In a typical CD-DeIB process, pure hydrogen is admixed with the C4 feed, or is fed to the tower at a lower point than the C4 feed. A hydroisomerization catalyst is incorporated in structures within the tower to affect the reaction. This type of CD-DeIB tower accomplishes several functions. First, it removes the isobutylene and isobutane from the feed, because they are undesirable as feed to the metathesis unit. Furthermore, this system hydroisomerizes 1-butene to 2-butene to improve recovery of 2-butene, since 1-butene has a boiling point close to that of isobutylene and tends to track overhead. A CD-DeIB tower also hydrogenates the small remaining amounts of butadiene after the selective hydrogenation, thereby reducing the butadiene content. Hydrogenation of butadiene is desirable because butadiene is a poison for the metathesis catalyst.

As indicated above, in a double bond hydroisomerization process, hydrogen must be co-fed with the C4 stream in order to keep the catalyst active. However, as a result, some of the butenes are saturated. This undesirable reaction leads to loss of valuable 2-butene feed for metathesis. It would be useful to develop an isomerization process in which the saturation rate of butenes to butanes is minimized.

### SUMMARY OF THE INVENTION

An object of the invention is to provide a double bond hydroisomerization process in which the conversion of 1-butene to 2-butene is improved over conventional processes.

Another object of the invention is to provide a butene double bond hydroisomerization process in which the production of butanes is minimized.

A further object of the invention is to provide a process for producing a metathesis feed stream containing high quantities of 2-butene with minimum losses of butenes to butanes.

Other objects will be in part obvious and in part pointed out more in detail hereafter.

A preferred form of the invention is a process for the double bond hydroisomerization of C₄ olefins, comprising obtaining a feed stream comprising 1-butene and 2-butene, introducing the feed stream and hydrogen to a reaction zone comprising a catalytic distillation column containing a hydroisomerization catalyst with double bond hydroisomerization activity in order to convert a portion of the 1-butene into 2-butene, forming a bottoms stream comprising 2-butene and a top stream comprising isobutane and isobutylene, and introducing carbon monoxide to the reaction zone in an amount of 0.001 to 0.03 moles of carbon monoxide per mole of hydrogen in order to increase the selectivity to 2-butene. In some cases, the feed stream includes butadiene, and at least a portion of the butadiene is hydrogenated to butene in the reaction zone.

In one embodiment, the reaction zone has an axial length and hydrogen is introduced to the reaction zone at multiple feed points along the axial length. Sometimes, both hydrogen and carbon monoxide are introduced to the reaction zone at multiple feed points along the axial length.

The catalyst typically is a group VIII metal, and often comprises palladium, platinum and/or nickel. Frequently, the catalyst is disposed on an alumina support.

Often, at least 75% of the 1-butene in the feed stream is hydroisomerized to 2-butene. In some cases, the molar ratio of 2-butene to 1-butene in the effluent stream is at least 85:15. In one embodiment, the molar ratio of 2-butene to 1-butene in the feed stream is no more than 80:20. The molar ratio of carbon monoxide to hydrogen introduced into the reaction zone frequently is in the range of 0.002 to 0.005.

In some cases, the process includes mixing the bottoms stream with a metathesis reactant to form a metathesis feed stream and introducing the metathesis feed stream to a metathesis reactor to form a metathesis product. Often, the metathesis reactant is ethylene and the metathesis product is propylene.

When the feed stream contains significant quantities of butadiene, the process further comprises hydrogenating the feed stream prior to introduction into the reaction zone in order to reduce the butadiene content of the feed stream.

Another embodiment is a process for the double bond hydroisomerization of C₄ olefins, comprising obtaining a feed stream comprising 1-butene and 2-butene, and introducing the feed stream and hydrogen to a reaction zone comprising a catalytic distillation column having a length and containing a catalyst with double bond hydroisomerization activity in order to convert a portion of the 1-butene into 2-butene, forming a bottoms stream comprising 2-butene and a top stream comprising isobutane and isobutylene, the hydrogen being introduced at multiple feed points along the length of the reaction zone in a quantity appropriate to maintain the catalyst in an active double bond hydroisomerization form while minimizing hydrogenation of butenes. Carbon monoxide also can be introduced into the reaction zone with hydrogen at one or more of the feed points along the length of the reactor.

Often, the process further comprises mixing the bottoms stream with a metathesis reactant to form a metathesis feed stream and introducing the metathesis feed stream to a metathesis reactor to form a metathesis product. When the feed stream contains substantial quantities of butadiene, the process often includes hydrogenating the feed stream prior to introduction into the reaction zone in order to reduce the butadiene content of the feed stream.

Yet another embodiment is an apparatus for the double bond hydroisomerization of 1-butene to 2-butene, comprising a C₄ feed stream conduit, a catalytic distillation column having a feed inlet fluidly connected to the olefin feed stream conduit, an upper end and a lower end, the catalytic distillation column containing a hydroisomerization catalyst, a first hydrogen inlet disposed on one of the C₄ feed stream conduit and the feed inlet, and a second hydrogen inlet disposed along the length of the catalytic distillation column above the feed inlet, the first and second hydrogen inlets being positioned to maintain a hydrogen content in the catalytic distillation column appropriate to maintain the hydroisomerization catalyst in an active double bond hydroisomerization form while minimizing hydrogenation of butenes. A hydrogenation reactor is sometimes disposed upstream from the catalytic distillation column. A metathesis reactor can be disposed downstream from the catalytic distillation column. The first and/or second hydrogen inlet can be configured to receive a mixture of hydrogen and carbon monoxide.

The invention accordingly comprises the several steps and the relation of one or more of such steps with respect to each of the others and the system possessing the features, properties, and the relation of elements exemplified in the following detailed disclosure.

### BRIEF DESCRIPTION OF THE DRAWINGS

FIG. 1 is a schematic drawing of a first embodiment of a process which employs a catalytic distillation-deisobutenizer (CD-DeIB) according to the invention.

FIG. 2 is a schematic drawing of a second embodiment of a process using a CD-DeIB with multiple stages of hydrogen or hydrogen/carbon monoxide feed according to the invention.

FIG. 3 is a schematic drawing of an embodiment in which a fixed bed reactor is used for double bond hydroisomerization with two stages of hydrogen or hydrogen/carbon monoxide feed.

FIG. 4 is a schematic drawing of an embodiment in which a fixed bed reactor is used for double bond hydroisomerization with three stages of hydrogen or hydrogen/carbon monoxide feed.

FIG. 5 is a schematic drawing of an embodiment in which a C4 feed stream is hydroisomerized in a CD-DeIB to produce a 2-butene stream which is subsequentaly fed to a metathesis reactor.

FIG. 6 is a schematic drawing of an embodiment in which a C4 feed stream is hydrogenated and hydroisomerized in a fixed bed reactor to produce a 2-butene feed stream which is subsequently fed to a metathesis reactor.

FIG. 7 is a schematic drawing of an embodiment in which a C4 feed stream is hydrogenated in a hydroigenation reactor and hydroisomerized in a catalytic distillation column to produce a 2-butene feed stream which is subsequently used in a metathesis process.

FIG. 8 is a schematic drawing of an embodiment in which a C4 feed stream is hydrogenated, hydroisomerized in a fixed bed reactor, and subjected to separation to produce a 2-butene feed stream which is subsequently used in a metathesis process.

FIG. 9 is a schematic drawing of an embodiment in which a C4 feed stream is hydrogenated, subjected to separation to remove isobutylene and/or isobutane, and then hydroisomerized in a fixed bed reactor to produce a 2-butene feed stream which is subsequently used in a metathesis process.

FIG. 10 is a graph showing the effect of hydrogen flow rate on butadiene conversion.

FIG. 11 is a graph showing the effect of hydrogen flow rate on 1-butene conversion and selectivity.

FIG. 12 is a graph showing the effect of multiple hydrogen injections on 1-butene conversion and selectivity.

FIG. 13 shows the effect of carbon monoxide and multiple hydrogen-carbon monoxide injections on butadiene conversion.

FIG. 14 shows the effect of carbon monoxide and multiple hydrogen-carbon monoxide injection on 1-butene conversion and selectivity.

### DETAILED DESCRIPTION OF THE INVENTION

The invention is an improved process for producing 2-butene by the hydroisomerization of normal C4 olefins in the presence of a particulate catalyst. The process produces minimal quantities of butane, which is an undesirable product, using two features that can be employed either separately or in combination. The first is co-feeding carbon monoxide (CO) with the hydrogen stream. The inventors have surprisingly found that CO acts as an inhibitor for the hydrogenation reactions while allowing the double bond hydroisomerization reactions to continue. The second technique is feeding the hydrogen or the hydrogen/CO mixture at one or more locations along the length of the reactor. Additionally, butadiene is hydrogenated to butenes.

Both features of the invention can be employed in gas-liquid fixed bed reactors as well as in catalytic distillation columns. The fixed bed reactors can be designed over any liquid-gas flow regimes, including those that generate pulsations. Upflow and downflow reactors can be employed. The use of a gas-liquid system enables moderate temperatures to be used, and allows for pumping, rather than compression, of the hydrocarbons. The reactor pressure range is usually between 2 and 30 barg, typically between 5 and 18 barg. The reactor inlet temperature range is usually between 80 and 250 F, typically 120 and 180 F. Carefully controlled hydrogen addition is used to avoid hydrogenation of butenes to butanes as described above. When a catalytic distillation column is used, the process makes use of the mass transfer resistance of hydrogen gas into liquid to keep the hydrogen concentration low in the reacting fluid and thus minimize hydrogenation of butenes to butanes.

When using a single injection of hydrogen and CO, the hydrogen and CO preferably are injected at a point upstream from the hydroisomerization reactor. In this case, the CO to H2 ratio is between 0.1% and 3% on a molar basis, more preferably 0.1-0.5%, and is typically 0.2-0.4% on a molar basis. When multiple injections of hydrogen and CO are used, the overall hydrogen/CO feed preferably is divided in order to provide that the total volume of the catalyst is in an active state. In this case, the CO and H2 preferably are injected together at multiple points along the length of the reactor. The ratio of CO to H2 at each point of injection preferably, but not necessarily, is the same as at the other points of injection. However, it is also feasible to have one of the streams contain only hydrogen. A portion, or all, of the hydrogen and/or CO can be mixed with the mixed C4 feed before the feed enters the hydroisomerization reactor.

It is well known that carbon monoxide is a reversible poison for Pd catalysts used in hydrogenation applications. It is believed that carbon monoxide will impede all reactions over that catalyst. The inventors have found, however, that when CO is used in the present invention at low levels to moderate hydrogenation activity, it will not impede double bond hydroisomerization but will selectively impede the hydrogenation reaction. Thus, its use will increase selectivity to isomerization. By adjusting the amount of CO and at the same time maintaining enough catalyst to achieve local isomerization, equilibrium improved isomerization/hydrogenation selectivity can be achieved.

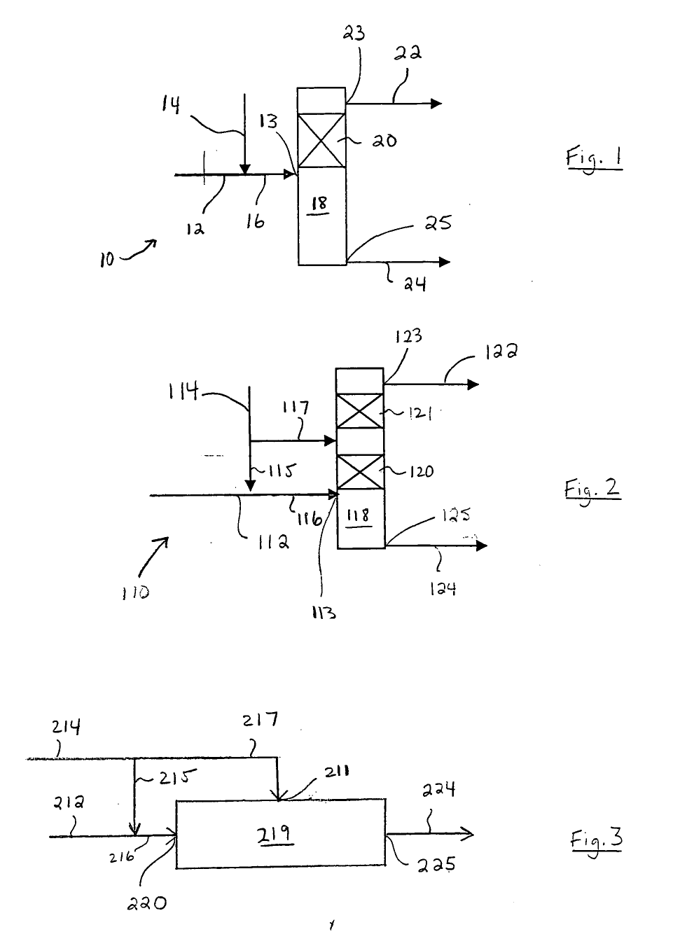

FIGS. 1 and 2 below show two options for a combined catalytic distillation-double bond hydroisomerization process. FIG. 1 depicts a system 10 for C4 double bond hydroisomerization with the injection of CO and hydrogen at a single point. A mixed C4 feed stream 12 is combined with a hydrogen-carbon monoxide gas stream 14 to form a catalytic distillation tower feed stream 16. The tower feed stream 16 enters the middle of a catalytic distillation tower 18 through inlet 13. The tower 18 has a reaction zone 20 above the feed point containing catalyst. The catalyst is located inside catalytic distillation structures or the structures are so designed that the materials have catalytic activity (e.g. an alumina distillation packing that has had catalyst impregnated therein). Isobutylene and isobutane are removed from the upper end of the tower 18 in top stream 22 through top outlet 23, along with most of the remaining 1-butene. In the reaction zone 20, 1-butene is hydroisomerized to 2-butene. The 2-butene is removed from the bottom of the tower 18 in bottom stream 24 through bottom outlet 25.

The catalyst employed in the double bond hydroisomerization process of the invention can be in the form of a typical particulate or shaped catalyst, or as a distillation packing. Catalyst which serves as distillation packing can be in a conventional distillation packing shape such as Raschig rings, pall rings, saddles or the like and as other structures such as, for example, balls, irregular shapes, sheets, tubes or spirals. The catalyst can be packed in bags or other structures, plated on grills or screens. Reticulated polymer foams can also be used as long as the structure of the foam is sufficiently large so as to not cause a high pressure drop through the column. Furthermore, it is important to have an appropriate rate of vapor flow through the column. A catalyst suitable for the present process is 0.4% PdO on ⅛″ Al2O3 (alumina) spheres, which is a double bond hydroisomerization catalyst supplied by Engelhard. Alternately other metals can be used including platinum and nickel, which can be either sulfided or unsulfided.

The catalytic distillation column pressure usually is between 2 and 12 barg, typically between 3 and 8 barg. The reactor inlet temperature usually is between 80 and 220 F, typically 100 and 160 F.

FIG. 2 shows hydrogen/CO injection into a catalytic distillation tower in which the hydrogen-CO stream is split into two separate inlet streams. The system is designated as 110. A mixed C4 feed stream 112 is combined with a first hydrogen-carbon monoxide gas stream 115, which is approximately half of gas stream 114, to form a catalytic distillation tower feed stream 116. This feed stream enters the middle of a catalytic distillation tower 118 through a lower inlet 113. The tower 118 has a lower reaction zone 120 above the feed point and an upper reaction zone 121 above the lower reaction zone 120. A second hydrogen-carbon monoxide gas stream 117 is fed to the tower 118 through upper inlet 111, which is located between the lower reaction zone 120 and the upper reaction zone 121. Isobutylene, isobutane and at least some of the remaining 1-butene are removed from the top of the tower in top stream 122. In the reaction zones 120 and 121, 1-butene is isomerized to 2-butene. The 2-butene is removed from the bottom of the tower in stream 124 through bottom outlet 125. It is also possible, but usually less desirable, for stream 115 and/or stream 117 to contain only hydrogen.

The hydrogenation reaction rate is a much stronger function of the hydrogen partial pressure than is the isomerization reaction rate. Using multiple hydrogen injection points along the length of the catalyst bed results in a local reduction in hydrogen concentration (i.e. a lower concentration at a particular point along the reactor length) as compared to an embodiment in which all of the hydrogen is introduced at the inlet to the reactor. This increases the isomerization/hydrogenation selectivity with and without the presence of CO.

FIG. 3 depicts an embodiment 210 which employs a fixed bed hydroisomerization reactor 219 and a hydrogen-carbon monoxide gas stream 214. Gas stream 214 is split into two streams of approximately equal flow rate, first gas stream 215 and second gas stream 217. A mixed C4 feed stream 212 is combined with the first gas stream 215 to form a reactor feed stream 216. The reactor feed stream 216 enters one end of the fixed bed hydroisomerization reactor 219 through inlet 220. The second gas stream 217 is fed to the reactor 219 a portion of the way along the length of the reactor 219 through inlet 211. Usually inlet 211 is ¼ to ½ of the way along the length of the reactor. In the reactor 219, 1-butene is isomerized to 2-butene. The reactor outlet stream 224 exits the reactor 219 through outlet 225. Stream 224 contains increased quantities of 2-butene as compared to prior known systems in which carbon monoxide is not used and/or all of the hydrogen is fed to the reactor 219 at a single location at the upstream end of the reactor 219. It is also possible, but usually less desirable, for stream 215 and/or stream 217 to contain only hydrogen.

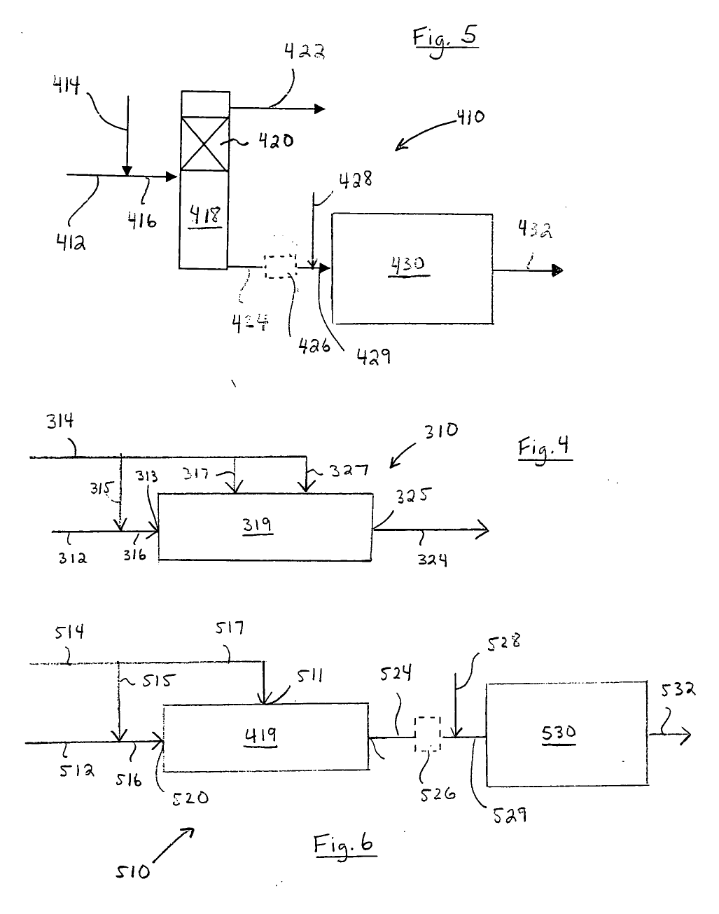

The embodiment of FIG. 4 is similar to that of FIG. 3 except that the FIG. 4 embodiment has three feed points for hydrogen and carbon monoxide. The system of FIG. 4 is designated as 310. The first hydrogen-carbon monoxide feed is in first gas stream 315, which combines with mixed C4 feed stream 312 to form reactor feed stream 316. First gas stream 315 has about one third of the flow rate of gas stream 314. Stream 316 enters the fixed bed hydroisomerization reactor 319 though inlet 313. The second gas stream 317, which typically constitutes another third of gas stream 314, is fed to the reactor 319 at a location about one third of the length from the reactor entrance. The third hydrogen-carbon monoxide stream 327, which is the remainder of gas stream 314, is fed to the reactor 319 at a location about one half to two thirds of the way along the length of the reactor 319. In the reactor 319, 1-butene is hydroisomerized to 2-butene, forming a reactor outlet stream 324 containing increased quantities of 2-butene. The reactor outlet stream 324 exits the reactor 319 through outlet 325. It is also possible, but usually less desirable, for one or more of stream 315, 317 and 327 to contain only hydrogen.

As is indicated above, the process of the invention is useful for the production of a butenes stream having a high concentration of 2-butenes. Preferably, the invention produces C4 streams in which the ratio of 2-butene to 1-butene is at least 8:1. This type of stream is a preferred feed for metathesis processes, as are shown in FIG. 5 and FIG. 6. In the embodiment of FIG. 5, designated as 410, a mixed C4 feed stream 412 is combined with a hydrogen-carbon monoxide stream 414 to form a catalytic distillation tower feed stream 416. The tower feed stream 416 enters the middle of a fractionation tower 418, which has a reaction zone 420 above the feed point. Isobutylene and isobutane are removed from the top of the tower in top stream 422 along with at least some of the remaining 1-butene. In the reaction zone 420, 1-butene is isomerized to 2-butene. The 2-butene is removed from the bottom of the tower in bottom stream 424. After optional removal of impurities in one or more guard beds 426, the bottom stream 424 is mixed with ethylene stream 428 to form a metathesis feed stream 429. The metathesis feed stream 429 enters the metathesis reactor 430, in which the ethylene and 2-butene react to form propylene. The propylene is removed from the metathesis reactor 430 in propylene stream 432.

FIG. 6 depicts a fixed bed hydroisomerization reactor similar to that shown in FIG. 3 upstream from a metathesis reactor. In this embodiment, designated as 510, hydrogen-carbon monoxide gas stream 514 is split into two streams of approximately equal flow rate, gas streams 515 and 517. A mixed C4 feed stream 512 is combined with the first hydrogen-carbon monoxide stream 515 to form a reactor feed stream 516. This feed stream 516 enters one end of a fixed bed hydroisomerization reactor 519 through inlet 520. The second hydrogen-carbon monoxide stream 517 is fed to the reactor 519 at a midpoint along the length of the reactor 519. In the reactor 519, 1-butene is isomerized to 2-butene. The reactor outlet stream 524 contains increased quantities of 2-butene as compared to prior known systems in which carbon monoxide is not used and/or all of the hydrogen is fed to the reactor 519 at a single location at the upstream end of the reactor 519. It is also possible, but usually less desirable, for stream 515 and/or stream 517 to contain only hydrogen. The reactor outlet stream 524 is optionally purified in one or more guard beds 526 and is mixed with an ethylene stream 528 to form a metathesis feed stream 529. Stream 529 enters a metathesis reactor 530, in which the 2-butene reacts with the ethylene to form a propylene stream 532.

By maximizing the 2-butene fraction using the processes shown in FIG. 5 and/or 6, one accomplishes several things. First, the yield of butenes in the hydrogenation/double bond hydroisomerization step is maximized because the loss of butenes to butanes (inerts in the metathesis process) is minimized. As a result, the production of propylene in a metathesis process using 2-butene and ethylene is maximized. Second, by maximizing the production of 2-butenes by double bond hydroisomerization, separation of the n-butenes from the isobutylene/isobutylene is facilitated, since 2-butene is heavier and has a higher boiling point than 1-butene, and is therefore more easily separated from isobutane and isobutylene in a fractionation process. Third, in the metathesis reaction, the reaction between 2-butene and ethylene maximizes propylene production. If 1-butene is present in the metathesis reactor, it will react with some of the 2-butene to produce C3s and C5s. Thus, the overall yield of C3s will be lower than if the 1-butene has been isomerized to 2-butene and reacts with ethylene to produce 2 C3s. It is noted that there is no reaction between 1-butene and ethylene.

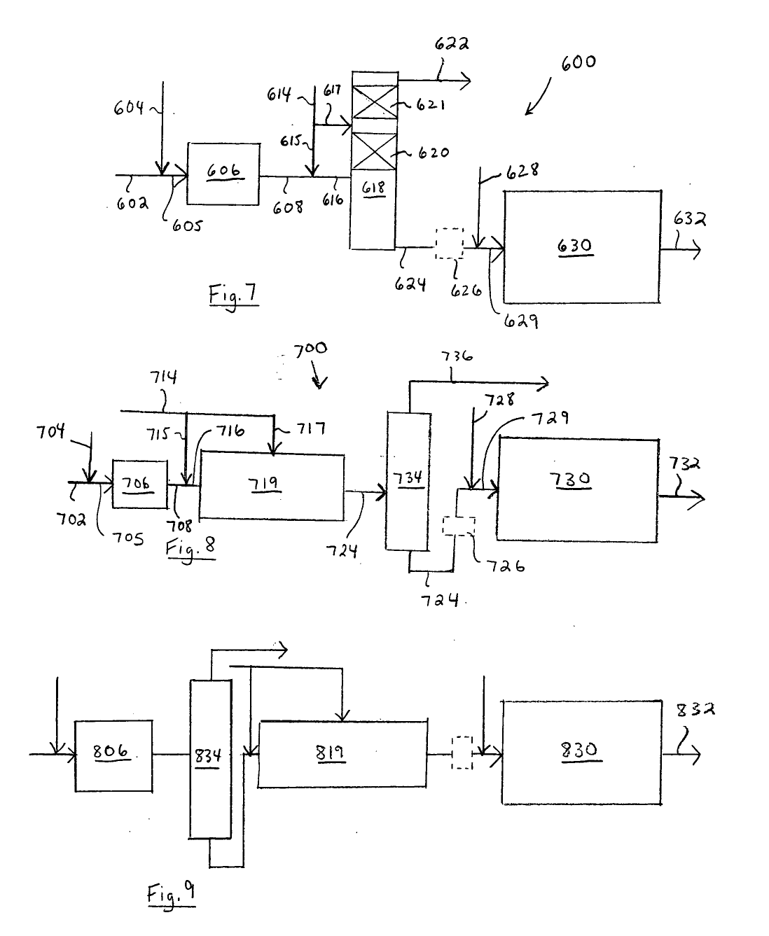

FIG. 7-9 depict embodiments in which a hydrogenation reactor is disposed upstream from a hydroisomerization reactor, and a metathesis reactor is positioned downstream from the hydroisomerization reactor. In FIG. 7, system 600 has a mixed C4 stream 602, which is combined with a hydrogen stream 604 to form a hydrogenation reactor feed stream 605. This stream is fed to a hydrogenation reactor 606 in which the butadiene content of mixture is reduced to about 1500 parts per million based on weight or less. The hydrogenation reactor effluent 608 is mixed with gas stream 615, which is half of hydrogen-carbon monoxide stream 614, and forms a catalytic distillation tower feed stream 616. This feed stream enters the middle of a catalytic distillation tower 618, which has a lower reaction zone 620 above the feed point and an upper reaction zone 621 above the lower reaction zone 620. A second hydrogen-carbon monoxide gas stream 617 is fed to the tower 618 at a location between the lower reaction zone 620 and the upper reaction zone 621. Isobutylene, isobutane and at least some of the remaining 1-butene are removed from the top of the tower in top stream 622. It is also possible, but usually less desirable, for stream 615 or stream 617 to contain only hydrogen. In the reaction zones 620 and 621, 1-butene is isomerized to 2-butene. The 2-butene is removed from the bottom of the tower in stream 624, optionally is purified in one or more guard beds 626, and mixed with an ethylene stream 628 to form a metathesis feed stream 629. Stream 629 enters the metathesis reactor 630, in which it is converted to propylene, which is removed from the metathesis reactor 630 in propylene stream 632.

FIGS. 8 and 9 depict a fixed bed hydroisomerization reactor downstream from a hydrogenation reactor and upstream from a metathesis reactor. In these embodiments, a fractionation column is included upstream or downstream from the hydroisomerization reactor in order to remove isobutane and/or isobutylene. In FIG. 8 the system 700 has a mixed C4 stream 702 which is combined with a hydrogen stream 704 to form a hydrogenation reactor feed stream 705. This stream is fed to a hydrogenation reactor 706 in which the butadiene content of mixture is reduced to about 1500 parts per million based on weight or less. The hydrogenation reactor effluent 708 is mixed with stream 715, which is half of hydrogen-carbon monoxide stream 714, forming a reactor feed stream 716. The reactor feed stream 716 enters one end of the fixed bed hydroisomerization reactor 719. The second hydrogen-carbon monoxide stream 717 is fed to the reactor 719 part way along the length of the reactor 719. In the reactor 719, 1-butene is isomerized to 2-butene. The reactor outlet stream 724 contains increased quantities of 2-butene as compared to prior known systems in which carbon monoxide is not used and/or all of the hydrogen is fed to the reactor 719 at a single location at the upstream end of the reactor 719. It is also possible, but usually less desirable, for stream 715 and/or stream 717 to contain only hydrogen. The reactor outlet stream 724 is fed to a fractionation column 734 in which isobutane and isobutylene are removed from the top in stream 736 and a 2-butene stream 724 is removed from the bottom. The 2-butene stream 724 is optionally purified in one or more guard beds 726 and is mixed an ethylene stream 728. The combined stream 729 enters a metathesis reactor 730, in which the 2-butene reacts with the ethylene to form a propylene stream 732.

The embodiment shown in FIG. 9 is similar to that of FIG. 8 with the exception that the fractionation column 834 for removing isobutane and isobutylene is upstream from the hydroisomerization reactor 819 and downstream from the hydrogenation reactor 806. The metathesis reactor 830 produces a propylene stream 832.

Having generally described the invention, the following examples are included for purposes of illustration so that the invention may be more readily understood and are in no way intended to limit the scope of the invention unless otherwise specifically indicated.

### EXAMPLE 1

### Injection of H2 or a CO—H2 Mixture at One Feed Point of a Catalytic Distillation Tower with No Butadiene in the C4 Feed Stream

A C4 double bond hydroisomerization and separation process was used to separate a C4 stream which did not contain butadiene. The reaction took place within a catalytic distillation tower fitted with both catalytic distillation structures and conventional inert distillation packing. The catalyst was 680 grams of 0.4% PdO on ⅛″ Al2O3 pellets (Engelhard) and was placed in bales wrapped in distillation wire mesh packing. The bales used covered 8 feet of a 2 in by 32 feet catalytic distillation tower (DC-100). The remainder of the tower was filled with ½ inch saddle packing.

The feed stream contained a mix of 2-butene, 1-butene and isobutylene. The composition of the feed stream is shown below on Table 1. The feed was introduced in the column below the entire 8 feet of catalyst.

TABLE 1

| | |
|---|---|
| n-Butane, wt % | 0.10 |
| 1-Butene, wt % | 17.36 |
| trans-2-Butene, wt % | 14.45 |
| cis-2-Butene, wt % | 8.74 |
| Isobutylene, wt % | 59.35 |
| 1,3 Butadiene, wt % | 0.00 |

Hydrogen (Examples 1A to 1C) and hydrogen/CO mixtures (Examples 1D-1E) were mixed with the feed before it was injected into the tower. In examples 1D-1E, the CO/H2 mole ratio was 0.003 or 0.3%. The feed rate in all cases was 4.5 lb/hr. The reflux ratio was set at 9.3. The liquid distillate product stream was continuously withdrawn. The distillate primarily contained the isobutylene in the feed, any unreacted 1-butene and a trace amount of 2-butenes. The quantity of 2-butene in the distilate was based on the fractionation efficiency. A bottoms stream consisting primarily of 2-butene was withdrawn from the tower. The normal butanes were split between the distillate overhead product and the bottoms product. A small nitrogen stream was fed to the overhead and was vented as required to maintain the pressure close to 80 psig.

Samples of the liquid distillate product and the bottoms were taken in gas bags or small steel bombs for analysis by gas chromatography using a flame ionization detector. Material balance runs were made by taking weighed samples of both distillate and bottoms over the same time period. The result of the experimental runs, which had varying top bed temperatures, CO flow rates, top reflux rates and bottoms flow rates are shown below in Table 2.

TABLE 2

| | Example Number | | | | |
|---|---|---|---|---|---|
| | 1A | 1B | 1C | 1D | 1E |
| Pressure, psig | 80 | 80 | 80 | 80 | 80 |
| Temperature Top of Bed (Deg. F.) | 129 | 130 | 131 | 131 | 129 |
| Total Feed rate, lbs/h | 4.5 | 4.5 | 4.5 | 4.5 | 4.5 |
| H2 to tower, stdcuft/h | 1.15 | 1.15 | 1.15 | 1.15 | 1.15 |
| CO in, as mol % of H2 in | 0.0 | 0.0 | 0.0 | 0.3 | 0.3 |
| CO to tower, stdcuft/h | 0.0 | 0.0 | 0.0 | 0.0035 | 0.0035 |
| Top reflux flow, lbs/h | 41.4 | 41.5 | 41.5 | 41.8 | 42.0 |
| LIQUID DISTILLATE Flow rate, lbs/hr | 3.01 | 3.04 | 3.48 | 3.68 | 3.32 |
| LIQUID DISTILLATE (wt % composition) | | | | | |
| n-Butane | 2.08 | 2.97 | 2.83 | 1.16 | 1.27 |
| 1-Butene | 8.76 | 8.87 | 9.80 | 8.15 | 7.34 |
| Trans-2-Butene | 5.59 | 10.96 | 13.99 | 19.20 | 15.22 |
| Cis-2-Butene | 0.50 | 1.10 | 1.45 | 2.20 | 1.68 |
| Isobutylene | 82.91 | 75.94 | 71.75 | 69.18 | 74.36 |
| 1,3 Butadiene | 0.00 | 0.00 | 0.00 | 0.00 | 0.00 |
| BOTTOMS PRODUCT Flow rate, lbs/hr | 1.39 | 1.09 | 0.98 | 0.74 | 0.98 |
| BOTTOMS PRODUCT (wt % composition) | | | | | |
| n-Butane | 1.15 | 0.90 | 0.59 | 0.20 | 0.28 |
| 1-Butene | 1.86 | 1.59 | 1.47 | 1.48 | 1.52 |
| Trans-2-Butene | 44.99 | 42.55 | 38.59 | 38.68 | 42.29 |
| Cis-2-Butene | 47.39 | 51.12 | 55.77 | 56.04 | 52.19 |
| Isobutylene | 4.62 | 3.84 | 3.59 | 3.60 | 3.72 |
| 1,3 Butadiene | 0.00 | 0.00 | 0.00 | 0.00 | 0.00 |
| 1-3 Butadiene % conversion | — | — | — | — | — |
| 1 Butene % conversion | 62.5 | 62.8 | 53.9 | 59.8 | 66.6 |
| 1 butene selectivity to butane, mol % | 14.8 | 19.0 | 23.1 | 8.5 | 7.7 |
| 1-butene converted, g/hr/g of catalyst | 0.332 | 0.334 | 0.286 | 0.318 | 0.356 |
| n-butane formed, g/hr/g of catalyst | 0.050 | 0.064 | 0.067 | 0.027 | 0.028 |

| | | |
|---|---|---|
| Average 1 butene selectivity to butane | 19.0 | 8.1 |
| Average 1 butene converted g/hr/g catalyst | 0.317 | 0.337 |
| Average 2 butenes formed g/hr/g catalyst | 0.257 | 0.310 |
| Average n butane formed g/hr/gm cat | 0.06 | 0.027 |

Table 2 shows the beneficial effect of using a mixture of hydrogen and carbon monoxide (0.3% CO to H2 molar ratio) for Examples 1D-1E instead of pure hydrogen for Examples 1A-1C. The selectivity of 1-butene to butane decreases from an average of 19% in Examples 1A-1C to about 8% in Examples 1D-1E while the overall rate of 1-butene conversion remains unchanged around the 60% level. The total 1 butene lost to butanes has decreased and the total production of 2 butenes has increased. As a result of the improved selectivity, the normal butane decreases from 3 wt % to 1 wt % in the liquid distillate stream and from 1 to 0.2% in the bottoms stream. At the same time the total amount of 1-butene converted varies only slightly between all examples, with the 2-butene in the bottoms ranging between 93 and 96% of the total C4s in the same stream for all the cases.

### EXAMPLE 2

### Injection of H2 or a CO—H2 Mixture at One Feed Point to a Catalytic Distillation Column with Butadiene in the C4 Feed Stream

The catalyst was loaded in the distillation column in a manner similar to that of Example 1. The column operation was the same as in Example 1. However, the feed included butadiene, as shown in Table 3, at 0.55% on a weight basis.

TABLE 3

| | |
|---|---|
| n-Butane, wt % | 0.09 |
| 1-Butene, wt % | 16.86 |
| trans-2-Butene, wt % | 14.45 |
| cis-2-Butene, wt % | 8.66 |
| Isobutylene, wt % | 59.39 |
| 1-3 Butadiene, wt % | 0.55 |

With butadiene in the feed, a higher hydrogen flow must occur in order to satisfy the requirement of hydrogen for butadiene hydrogenation to butenes while maintaining hydrogen to facilitate the hydroisomerization reaction. The feed rate and reflux remains the same as for the case in example 1. Table 4 shows the effect of using a mixture of hydrogen and carbon monoxide (0.3% CO to H2 molar ratio) for Examples 2D-2F instead of pure hydrogen for Examples 2A-2C, when butadiene is present.

TABLE 4

| | Example | | | | | |
|---|---|---|---|---|---|---|
| | 2A | 2B | 2C | 2D | 2E | 2F |
| Pressure, psig | 80 | 80 | 80 | 80 | 80 | 80 |
| Temperature Top of Bed (F.) | 129 | 128 | 130 | 130 | 129 | 128 |
| Tot Feed rate, lbs/h | 4.48 | 4.48 | 4.48 | 4.50 | 4.50 | 4.48 |
| H2 to DC-100, scfh | 1.15 | 1.15 | 1.15 | 1.15 | 1.15 | 1.15 |
| CO in, mol % of H2 in | 0.00 | 0.00 | 0.00 | 0.30 | 0.30 | 0.30 |
| CO to DC-100, scfh | 0 | 0 | 0 | 0.0035 | 0.0035 | 0.0035 |
| TRFX flow, lbs/h | 41.9 | 41.9 | 41.7 | 41.8 | 42.3 | 42.1 |
| LIQUID DISTILLATE Flow rate, lbs/hr | 3.55 | 2.89 | 4.33 | 4.15 | 3.46 | 2.67 |
| LIQUID DISTILLATE (wt % composition) | | | | | | |
| n-Butane | 2.77 | 2.35 | 3.08 | 0.83 | 0.77 | 0.67 |
| 1-Butene | 8.05 | 8.93 | 9.12 | 8.76 | 8.73 | 8.77 |
| Trans-2-Butene | 10.54 | 7.66 | 13.58 | 15.27 | 12.90 | 10.18 |
| Cis-2-Butene | 1.15 | 0.66 | 1.46 | 1.81 | 1.42 | 1.03 |
| Isobutylene | 77.34 | 80.23 | 72.61 | 73.24 | 76.09 | 79.26 |
| 1,3 Butadiene | 0.00 | 0.00 | 0.00 | 0.00 | 0.00 | 0.00 |
| BOTTOMS PRODUCT Flow rate, lbs/hr | 0.90 | 1.31 | 0.61 | 0.68 | 1.01 | 1.29 |
| BOTTOMS PRODUCT (wt % composition) | | | | | | |
| n-Butane | 0.93 | 0.93 | 0.72 | 0.15 | 0.16 | 0.18 |
| 1-Butene | 1.66 | 1.46 | 1.48 | 1.46 | 1.66 | 1.85 |
| Trans-2-Butene | 43.65 | 43.18 | 41.19 | 39.10 | 39.14 | 39.48 |
| Cis-2-Butene | 49.63 | 49.15 | 53.02 | 55.79 | 54.86 | 53.94 |
| Isobutylene | 4.07 | 5.22 | 3.55 | 3.46 | 4.13 | 4.50 |
| 1,3 Butadiene | 0.05 | 0.07 | 0.03 | 0.04 | 0.05 | 0.05 |
| 1-3 Butadiene % conversion | 98.2 | 96.5 | 99.1 | 99.0 | 97.8 | 97.2 |
| 1 Butene % conversion | 55.9 | 63.3 | 46.5 | 49.4 | 57.3 | 65.2 |
| 1 butene selectivity to butane, mol % | 25.9 | 15.3 | 36.7 | 8.3 | 5.4 | 3.2 |
| 1-butene converted, g/hr/g of catalyst | 0.288 | 0.325 | 0.239 | 0.251 | 0.295 | 0.335 |
| n-butane formed, g/hr/g of catalyst | 0.076 | 0.050 | 0.089 | 0.021 | 0.016 | 0.011 |

| | | |
|---|---|---|
| Average 1 butene selectivity to butane | 26 | 5.6 |
| Average 1 butene converted g/hr/gm catalyst | 0.284 | 0.294 |
| Average 2 butenes formed g/hr/g catalyst | 0.196 | 0.262 |
| Average n butane formed g/hr/gm cat | 0.072 | 0.016 |

While the requirements for hydrogenation have changed with the addition of butadiene, the positive influence of CO addition in the hydrogen is evident. The butadiene conversion is high (between 96 to 99%) with and without the presence of CO. There is sufficient hydrogen available to allow the hydrogenation of butadiene in this case without increasing the hydrogen above that used for Example 1. For all the cases there is 0 ppm butadiene in the liquid distillate product and between 300 and 700 ppm butadiene in the bottoms product. All the butadiene forced over the catalyst section by separation is essentially converted to butenes. The addition of the butadiene causes a drop in the average grams of 1-butene converted. The amount of 1-butene converted in Example 2 is approximately 88% of the 1-butene converted in Example 1. This is as expected since butadiene would be the first to react over this catalyst. However, the total amount of 1-butene converted to 2-butene by isomerization or butane by saturation remains high after the introduction of CO. This indicates that for all the cases there is enough hydrogen to both achieve butadiene hydrogenation and keep the catalyst active for 1-butene isomerization.

The presence of CO suppresses the undesirable 1-butene hydrogenation reaction. The selectivity of 1-butene to butane drops from an average of 26.0% for examples 2A-2C down to an average of 5.6% for Examples 2D-2F. As a result of the improved selectivity, the normal butane decreases from 3 wt % to less than 1 wt % in the liquid distillate stream and from 1 to 0.2% in the bottoms stream.

### EXAMPLE 3

### Injection of H2 at Multiple Feed Points with No Butadiene in the C4 Feed Stream

The catalyst was loaded in the distillation column in a manner similar to that of Example 1. The column operation also remained the same. No butadiene or CO was present in this example and the feed was that shown on Table 1. However, in Example 3B, the hydrogen flow was split equally between two separate injection ports. The bottom injection point is the same as that of Example 3A, i.e., together with the C4 feed. The second injection point is in the middle of the tower, with 4 feet of catalyst below and four feet of catalyst above it. Table 5 shows the effect of splitting the hydrogen while keeping the total hydrogen flow rate constant.

TABLE 5

Example Number

| | 3A | 3B |
|---|---|---|
| Pressure, psig | 80 | 80 |
| Temperature Top of Bed (Deg. F.) | 131 | 129 |
| Total Feed rate, lbs/h | 4.5 | 4.5 |
| H2 to Bottom column, scf/h | 1.15 | 0.58 |
| H2 to Top column, scf/h | 0.00 | 0.58 |
| CO in, as mol % of H2 in | 0.0 | 0.0 |
| CO to tower, stdcuft/h | 0.0 | 0.0 |
| Top reflux flow, lbs/h | 41.5 | 41.8 |
| LIQUID DISTILLATE Flow rate, lbs/hr | 3.48 | 3.06 |
| LIQUID DISTILLATE (wt % composition) | | |
| n-Butane | 2.83 | 1.99 |
| 1-Butene | 9.80 | 9.96 |
| Trans-2-Butene | 13.99 | 9.28 |
| Cis-2-Butene | 1.45 | 0.84 |
| Isobutylene | 71.75 | 77.75 |
| 1,3 Butadiene | 0.00 | 0.00 |
| BOTTOMS PRODUCT Flow rate, lbs/hr | 0.98 | 1.26 |
| BOTTOMS PRODUCT (wt % composition) | | |
| n-Butane | 0.59 | 0.20 |
| 1-Butene | 1.47 | 1.80 |
| Trans-2-Butene | 38.59 | 39.5 |
| Cis-2-Butene | 55.77 | 54.2 |
| Isobutylene | 3.59 | 4.50 |
| 1,3 Butadiene | 0.00 | 0.00 |
| 1-3 Butadiene % conversion | — | — |
| 1 Butene % conversion | 53.9 | 57.62 |
| 1 butene selectivity to butane, mol % | 23.1 | 11.83 |
| 1-butene converted, g/hr/g of catalyst | 0.286 | 0.306 |
| Average 2 butenes formed g/hr/g catalyst | 0.219 | 0.269 |
| n-butane formed, g/hr/g of catalyst | 0.067 | 0.037 |

When multiple points of hydrogen injection were used in place of a single point of injection, the selectivity of 1-butene to butane decreased from 23% in the cases with a single hydrogen injection to 11.8% for the cases with split hydrogen, while overall 1-butene conversion remains essentially unchanged. As a result, the n-butane decreased from about 3 wt % to about 2 wt % in the liquid distillate stream and from 0.6 wt % to 0.2 wt % in the bottoms stream. At the same time the total amount of 1-butene converted changed only slightly, with the 2-butene in the bottoms varying between 94% and 96% of the total C4's in the same stream for all the cases.

### COMPARATIVE EXAMPLE 4

### Fixed Bed Hydroisomerization Reactor with Single Point of Hydrogen Injection

A trickle bed reactor model was used to determine the benefits of multiple hydrogen injections and combined hydrogen-carbon monoxide injections in a hydroisomerization reactor. The reaction kinetics used for this calculation are consistent with catalytic distillation results from Examples 1 to 3. In this Comparative Example, a single point of hydrogen injection was used at three different hydrogen flow rates to determine the effect of hydrogen flow rate on butadiene conversion. Hydrogen flow rates were based upon hydrogen to butadiene molar ratios. Ratios of 2, 5 and 10 were used. All the results reflect a condition of 100% catalyst wetting and minimal pressure drop through the reactor. The heat balance calculation was based on an adiabatic reactor with vaporization. The composition of the feed is shown below on Table 6.

TABLE 6

| | Feed wt % |
|---|---|
| Butadiene | 0.13 |
| 1-butene | 11.00 |
| 2-butene | 26.00 |
| isobutane | 29.00 |
| isobutylene | 19.00 |
| n-butane | 14.87 |

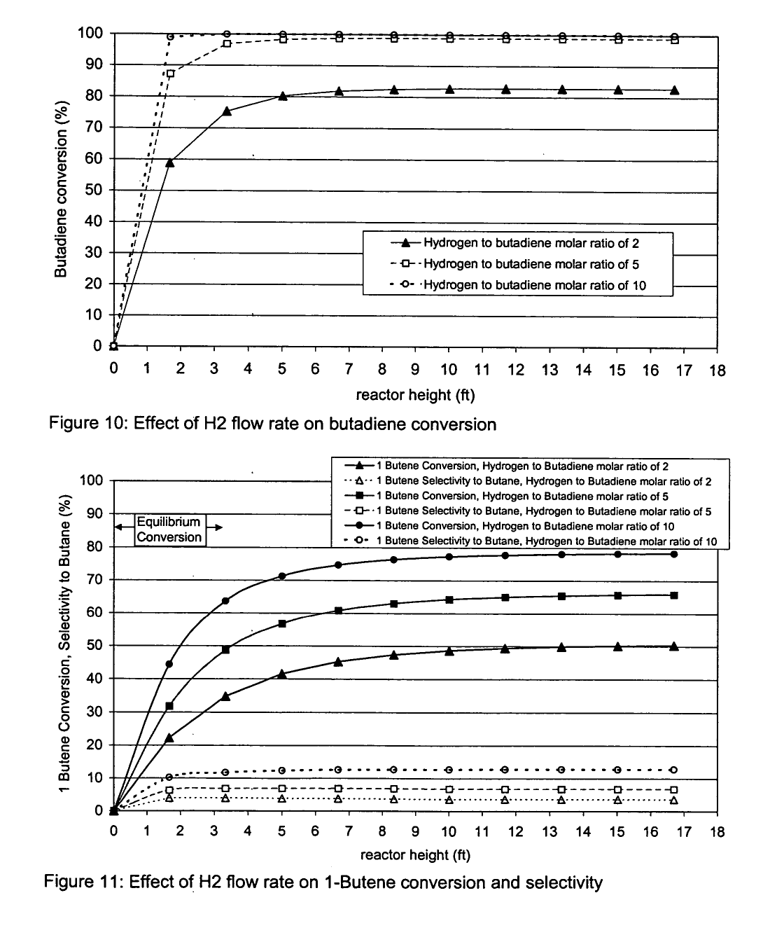

The inlet T of the reactor was set to 140 deg. F. and a pressure of 240 psig. The flow rate of C4s was 88,000 lbs/hour. The effect of the hydrogen flow rate on butadiene conversion is shown in FIG. 10. The effect of the hydrogen flow rate on 1-butene conversion and selectivity is shown in FIG. 11.

The equilibrium 1-butene conversion assuming no losses for this example is 86%. Based on FIGS. 10 and 11, butadiene conversions in excess of 99% can be achieved as long as the H2 to butadiene mole ratio is at least 5. Lowering the H2 feed rate to a ratio of 2 provides good selectivities but at the expense of both butadiene and 1-butene conversions. Increasing the H2 ratio to 10 leads to much higher losses and 13% selectivity of 1-butene to butane. For all these cases a reactor height of 10 feet was sufficient to handle most (about 98%) of the maximum 1-butene conversion. A H2-butadiene ratio of 5 and a reactor length of 10 ft gave a 65% 1-butene conversion at 6.7% selectivity to butane and with 15 ppmw butadiene at the outlet.

### EXAMPLE 4

### Fixed Bed Hydroisomerization Reactor with Split H2 Injection

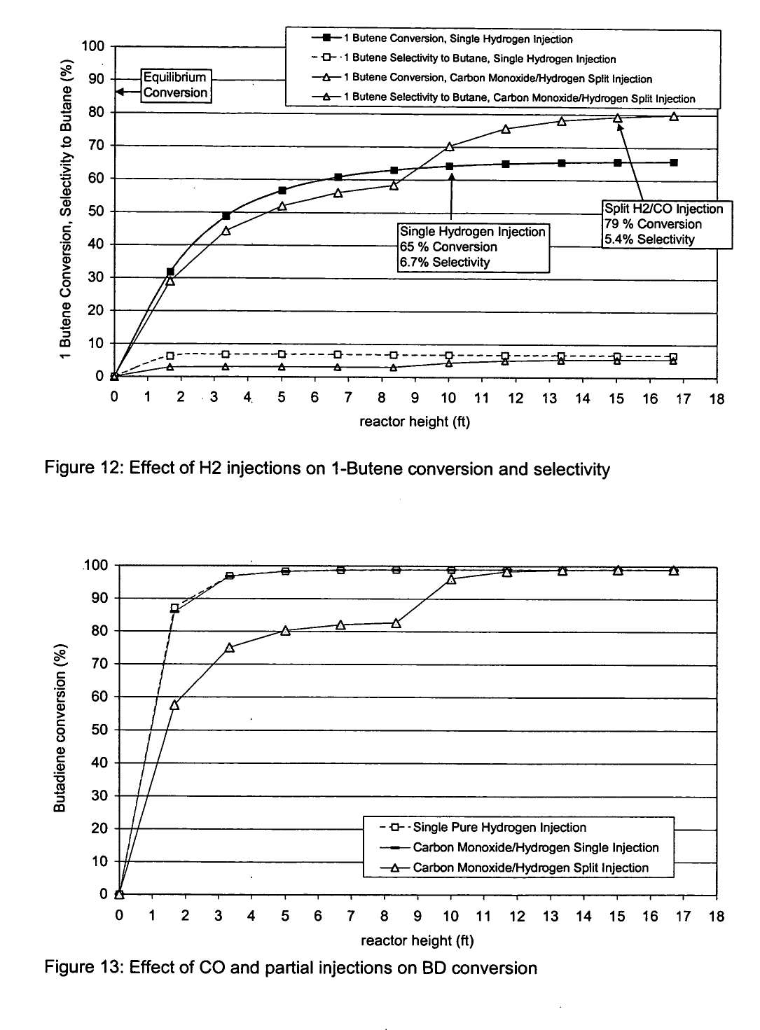

Comparative Example 4 was repeated using a hydrogen to butadiene mole ratio of 5 with the exception that the hydrogen was split into two separate feeds to the hydroisomerization reactor. Due to the higher dependence of hydrogenation rate to hydrogen partial pressure relative to the isomerization rate a low H2 partial pressure throughout the reactor was expected to be beneficial to selectivity. In this Example the total H2 rate was kept constant at a H2 to butadiene mole ratio of 5, and the gas flow was evenly split with half of it coming in with the feed and the other half injected at 8 ft. along the reactor. The performance difference is provided in FIG. 12.

An improvement in 1-butene conversion (72%) was obtained while lowering the selectivity (6%) to butane. At the same time butadiene in the outlet was 13 ppmw.

### EXAMPLE 5

### Fixed Bed Hydroisomerization Reactor with Single and Split Injections of Hydrogen-Carbon Monoxide

A combined hydrogen-carbon monoxide stream was injected at a single point and in a split injection in a simulation of a fixed bed reactor using the C4 feed stream of Comparative Example 4. The CO to hydrogen mole ratio was 0.3%. The hydrogen to butadiene mole ratio was 5. The kinetic constants for butadiene and 1-butene hydrogenation were halved with the presence of a CO/H2 mixture of 0.3% mole based on the results of Example 2.

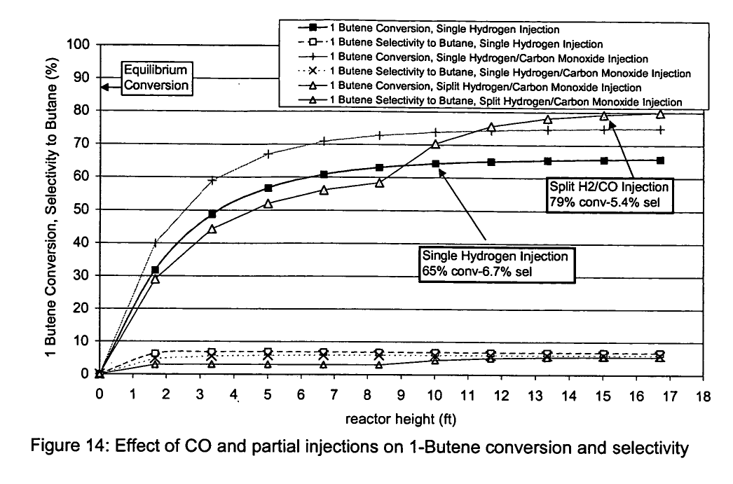

For the split feed embodiment, the second injection made was 8 ft from the reactor entrance. FIG. 13 shows the effect of a single hydrogen-carbon monoxide feed and a split hydrogen-carbon monoxide feed on butadiene conversion. FIG. 14 shows the effects of a single hydrogen-carbon monoxide feed and a split hydrogen-carbon monoxide feed on 1-butene conversion and selectivity. Combining the both inclusion of carbon monoxide and use of a split feed provides the optimized reactor case in FIG. 14 with 79% conversion and only 5.4% selectivity to butane. The butadiene at the reactor outlet is 13 ppmw. A substantial improvement was achieved with the combination of H2-CO in split injections going from 65% 1-butene conversion at 6.7% selectivity in Comparative Example 4 to 79% conversion and only 5.4% selectivity to butane in Example 5. The results showing the effect of CO are summarized in Table 7.

TABLE 7

| | Catalyst | | 1-Butene | 1-Butene |
|---|---|---|---|---|
| | Volume | ppm BD | conversion | sel. to |
| | (ft³) | outlet | (%) | butane (%) |
| Pure H2 one injection | 160 | 14 | 65 | 6.7 |
| H₂/BD ratio = 5 | | | | |
| Split H₂ and CO case | 160 | 14 | 74 | 6.0 |
| one injection | | | | |
| Split H₂ and CO case | 240 | 13 | 79 | 5.4 |

As will be apparent to persons skilled in the art, various modifications and adaptations of the method and structure above described will become readily apparent without departure from the spirit and scope of the invention, the scope of which is defined in the appended claims.

What is claimed is:

1. A process for the double bond hydroisomerization of C₄ olefins, comprising:
obtaining a feed stream comprising 1-butene and 2-butene,
introducing said feed stream and hydrogen to a reaction zone comprising a catalytic distillation column containing a hydroisomerization catalyst with double bond hydroisomerization activity in order to convert a portion of said 1-butene into 2-butene, forming a bottoms stream comprising 2-butene and a top stream comprising isobutane and isobutylene, and
introducing carbon monoxide to said reaction zone in an amount of 0.001 to 0.03 moles of carbon monoxide per mole of hydrogen in order to increase the selectivity to 2-butene.

2. The process of claim 1, wherein said feed stream includes butadiene.

3. The process of claim 2, wherein at least a portion of said butadiene is hydrogenated to butene in said reaction zone.

4. The process of claim 1, wherein said reaction zone has an axial length and hydrogen is introduced to said reaction zone at multiple feed points along said axial length.

5. The process of claim 1, wherein said reaction zone has an axial length and both hydrogen and carbon monoxide are introduced to said reaction zone at multiple feed points along said axial length.

6. The process of claim 1, wherein said catalyst comprises at least one member selected from the group consisting of palladium, platinum and nickel.

7. The process of claim 6, wherein said catalyst is disposed on an alumina support.

8. The process of claim 1, wherein said feed stream further contains normal butanes, isobutane, isobutene and butadiene.

9. The process of claim 1, wherein at least 75% of said 1-butene in said feed stream is hydroisomerized to 2-butene.

10. The process of claim 1, wherein the molar ratio of 2-butene to 1-butene in said effluent stream is at least 85:15.

11. The process of claim 10, wherein the molar ratio of 2-butene to 1-butene in said feed stream is no more than 80:20.

12. The process of claim 1, wherein the molar ratio of carbon monoxide to hydrogen introduced into said reaction zone is in the range of 0.002 to 0.005.

13. The process of claim 1, further comprising mixing said bottoms stream with a metathesis reactant to form a metathesis feed stream and introducing said metathesis feed stream to a metathesis reactor to form a metathesis product.

14. The process of claim 13, wherein said metathesis reactant is ethylene and said metathesis product is propylene.

15. The process of claim 1, wherein said feed stream contains butadiene, further comprising hydrogenating said feed stream prior to introduction into said reaction zone in order to reduce the butadiene content of said feed stream.

16. The process of claim 13, wherein said feed stream contains butadiene, further comprising hydrogenating said feed stream prior to introduction into said reaction zone in order to reduce the butadiene content of said feed stream.

17. A process for the double bond hydroisomerization of C₄ olefins, comprising:
obtaining a feed stream comprising 1-butene and 2-butene, and
introducing said feed stream and hydrogen to a reaction zone comprising a catalytic distillation column having a length and containing a catalyst with double bond hydroisomerization activity in order to convert a portion of said 1-butene into 2-butene, forming a bottoms stream comprising 2-butene and a top stream comprising isobutane and isobutylene, said hydrogen being introduced at multiple feed points along said length of said reaction zone in a quantity appropriate to maintain said catalyst in an active double bond hydroisomerization form while minimizing hydrogenation of butenes.

18. The process of claim 17, wherein carbon monoxide is introduced into said reaction zone with hydrogen at one or more of said feed points along said length of said reactor.

19. The process of claim 17, further comprising mixing said bottoms stream with a metathesis reactant to form a metathesis feed stream and introducing said metathesis feed stream to a metathesis reactor to form a metathesis product.

20. The process of claim 19, wherein said metathesis reactant is ethylene and said metathesis product is propylene.

21. The process of claim 17, wherein said feed stream contains butadiene, further comprising hydrogenating said feed stream prior to introduction into said reaction zone in order to reduce the butadiene content of said feed stream.

22. The process of claim 19, wherein said feed stream contains butadiene, further comprising hydrogenating said feed stream prior to introduction into said reaction zone in order to reduce the butadiene content of said feed stream.

23. An apparatus for the double bond hydroisomerization of 1-butene to 2-butene, comprising:
a C₄ feed stream conduit,
a catalytic distillation column having a feed inlet fluidly connected to said olefin feed stream conduit, an upper end and a lower end, said catalytic distillation column containing a hydroisomerization catalyst,
a first hydrogen inlet disposed on one of said C₄ feed stream conduit and said feed inlet, and
a second hydrogen inlet disposed along said length of said catalytic distillation column above said feed inlet, said first and second hydrogen inlets being positioned to maintain a hydrogen content in said catalytic distillation column appropriate to maintain said hydroisomerization catalyst in an active double bond hydroisomerization form while minimizing hydrogenation of butenes.

24. The apparatus of claim 23, further comprising:
a hydrogenation reactor disposed upstream from said catalytic distillation column.

25. The apparatus of claim 23, further comprising:
a metathesis reactor disposed downstream from said catalytic distillation column.

26. The apparatus of claim 23, wherein at least one of said first hydrogen inlet and said second hydrogen inlet is configured to receive a mixture of hydrogen and carbon monoxide.
